(19) United States
(12) Patent Application Publication
(10) Pub. No.: US 2017/0367397 A1
(43) Pub. Date: Dec. 28, 2017

(54) INFANT FORMULA COMPRISING HUMAN MILK OLIGOSACCHARIDES, POLYUNSATURATED FATTY ACIDS, NUCLEOTIDES, AND LUTEIN

(71) Applicant: ABBOTT LABORATORIES, Abbott Park, IL (US)

(72) Inventors: DANIEL ALBRECHT, Columbus, OH (US); MARK ENGLE, Plain City, OH (US)

(73) Assignee: ABBOTT LABORATORIES, Abbott Park, IL (US)

(21) Appl. No.: 15/531,034

(22) PCT Filed: Nov. 25, 2015
(86) PCT No.: PCT/US2015/062721

§ 371 Date: May 26, 2017

Related U.S. Application Data
(60) Provisional application No. 62/084,705, filed on Nov. 26, 2014.

(51) Int. Cl. A23L 33/00; A61K 31/702; A61K 31/355; A61K 31/202; A23L 33/12; A23L 33/10; A61K 9/00; A23L 33/13; A23L 33/17; A61K 31/708; A61K 31/047

(52) U.S. Cl. CPC A23L 33/40; A61K 31/708; A61K 31/702; A61K 31/355; A61K 31/202; A61K 31/047; A23L 33/10; A61K 9/0095; A23L 33/30; A23L 33/13; A23L 33/17; A23L 33/12; A23V 2002/00

(57) ABSTRACT

Infant formulas that include at least one human milk oligosaccharide selected from 2′-fucosyllactose (2′-FL) and lacto-N-neotetraose (LNnT), polyunsaturated fatty acids (PUFAs) docosahexaenoic acid (DHA) and arachidonic acid (AA), where the AA and DHA are present in an n-6/n-3 ratio of between 1 and 1.8, at least 30 mg/L of nucleotides, where the nucleotides include inosine monophosphate, and at least 30 μg/L of lutein are disclosed. Also provided are methods of use of the infant formulas for improving at least one of cognition, learning, and memory in an infant. The methods include administering the infant formulas to an infant.

### CROSS REFERENCE TO RELATED APPLICATIONS

This application claims priority to and the benefit of U.S. Application No. 62/084,705, filed on Nov. 26, 2014, the entire content of which is incorporated herein by reference.

### FIELD

The present disclosure relates to infant formulas and methods of use of the infant formulas for improving at least one of cognition, learning, and memory in an infant. In particular, the infant formulas include at least one human milk oligosaccharide selected from 2′-fucosyllactose (2′-FL) and lacto-N-neotetraose (LNnT), polyunsaturated fatty acids (PUFAs) docosahexaenoic acid (DHA) and arachidonic acid (AA), where the AA and DHA are present in an n-6/n-3 ratio of between 1 and 1.8, at least 30 mg/L of nucleotides, where the nucleotides include inosine monophosphate, and at least 30 μg/L of lutein.

### BACKGROUND

Infant formulas are commonly used today to provide a supplemental or sole source of nutrition early in life to both preterm and term infants. These formulas typically contain basic nutrients for the infant's diet, and are commercially available as powders, ready-to-feed liquids, and liquid concentrates. Many infant formulas provide a quality alternative to human milk, as not all infants receive human milk.

Infancy is a time when development of both the central and peripheral nervous system occurs. Including ingredients in an infant formula that promote nervous system development, function, or both, improves one or more of cognition, learning, and memory in an infant.

### SUMMARY

The present disclosure is directed to infant formulas that include a combination of at least one human milk oligosaccharide selected from 2′-fucosyllactose (2′-FL) and lacto-N-neotetraose (LNnT), polyunsaturated fatty acids (PUFAs) docosahexaenoic acid (DHA) and arachidonic acid (AA), where the AA and DHA are present in an n-6/n-3 ratio of between 1 and 1.8, at least 30 mg/L of nucleotides, where the nucleotides include inosine monophosphate, and at least 30 μg/L of lutein.

Also provided are methods of use of the infant formulas for improving at least one of cognition, learning, and memory in an infant. The methods include administering to the infant an infant formula as disclosed herein.

The infant formulas and methods of the present disclosure offer an alternative therapeutic or nutritional intervention option that can contribute to one or more of improved cognition, learning, and memory in individuals, and particularly in infants, toddlers, and children.

### DETAILED DESCRIPTION

The present disclosure is directed to infant formulas that include a combination of at least one human milk oligosaccharide selected from 2′-fucosyllactose (2′-FL) and lacto-N-neotetraose (LNnT), polyunsaturated fatty acids (PUFAs) docosahexaenoic acid (DHA) and arachidonic acid (AA), where the AA and DHA are present in an n-6/n-3 ratio of between 1 and 1.8, at least 30 mg/L of nucleotides, where the nucleotides include inosine monophosphate, and at least 30 μg/L of lutein.

The term “infant” as used herein, refers generally to individuals up to age 36 months of age, actual or corrected, including individuals up to age 12 months of age, actual or corrected.

The term “preterm infant” as used herein refers to those infants born at less than 37 weeks gestation, have a birth weight of less than 2500 gm, or both.

As used herein, all concentrations expressed as either “μg/liter” or “mg/liter” refer to ingredient concentrations within the described infant formulas as calculated on an as-fed basis, unless otherwise specified.

The term “cognition” as used herein, unless otherwise specified, refers to the learning, thinking, and memory (i.e., memory acquisition, memory retention and memory recall) of the brain. The term “improving cognition” as used herein, unless otherwise specified, refers to improving at least one of the learning, thinking, and memory functions of an infant.

The term “infant formula” as used herein, unless otherwise specified, refers to liquid and solid nutritional products suitable for consumption by an infant as a main source of nutrition. The term “infant formula” does not include human breast milk.

All percentages, parts and ratios as used herein, are by weight of the total composition, unless otherwise specified. All such weights as they pertain to listed ingredients are based on the active level and, therefore, do not include solvents or by-products that may be included in commercially available materials, unless otherwise specified.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range.

The various embodiments of the infant formulas of the present disclosure may include trace amounts of any optional or selected essential ingredient or feature described herein, provided that the remaining formulation still contains all of the required ingredients or features described herein. In this context, and unless otherwise specified, the term “trace amount” means that the selected formulation contains no more than 2 wt % of the optional ingredient, typically less than 1 wt %, and also includes zero percent, of such optional or selected essential ingredient, by weight of the infant formula.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the infant formulas of the present disclosure may also be substantially free of any ingredient or feature described herein, provided that the remaining formula still contains all of the required ingredients or features as described herein. In this context, and unless otherwise specified, the term “substantially free” means that the selected composition contains less than a functional amount of the optional ingredient, typically less than 1%, including less than 0.5%, including less than 0.1%, and also including zero percent, by weight of such optional or selected essential ingredient.

The infant formulas and corresponding manufacturing methods may comprise, consist of, or consist essentially of the elements of the products as described herein, as well as any additional or optional element described herein or otherwise useful in nutritional product applications.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

### Human Milk Oligosaccharides 2′-Fucosyllactose (2′-FL) and Lacto-N-Neotetraose (LNnT)

The infant formulas described herein include at least one of the human milk oligosaccharides (HMOs) 2′-FL and LNnT. Research has shown multiple roles for HMOs in improvement of central nervous system (CNS) function. For example, HMOs have been shown to stimulate enteric nerve cells, to reduce inflammation, including neuroinflammation, to promote the growth of beneficial microorganisms, and to increase levels of neuroprotective compounds. It is applicants' belief that inclusion of at least one of the HMOs 2′-FL and LNnT in the infant formula can improve neurological functions, such as cognition, learning, and memory, associated with the CNS.

In certain aspects, the 2′-FL, the LNnT, or both, are present in the infant formula at a concentration effective to improve one or more of cognition, learning, and memory in an infant, including a concentration from about 0.0001 mg/mL to about 50 mg/mL, from about 0.001 mg/mL to about 20 mg/mL, from about 0.01 mg/mL to about 10 mg/mL, from about 0.05 mg/mL to about 5 mg/mL, from about 0.5 mg/mL to about 1 mg/mL, or at a concentration of about 0.0001 mg/mL, about 0.0005 mg/mL, about 0.001 mg/mL, about 0.005 mg/mL, about 0.01 mg/mL, about 0.025 mg/mL, about 0.05 mg/mL, about 0.075 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.2 mg/mL, about 1.4 mg/mL, about 1.6 mg/mL, about 1.8 mg/mL, about 2.0 mg/mL, about 5.0 mg/mL, about 7.5 mg/mL, about 10 mg/mL, about 15 mg/mL, about 20 mg/mL, about 25 mg/mL, about 30 mg/mL, about 40 mg/mL, or about 50 mg/mL.

In addition to including at least one of the 2′-FL and the LNnT described above, in certain aspects, the infant formula includes additional sialylated or fucosylated human milk oligosaccharides (HMOs). Any or all of the HMO(s) used in the infant formula may be isolated or enriched from milk(s) secreted by mammals including, but not limited to: human, bovine, ovine, porcine, or caprine species. The HMOs may also be produced via microbial fermentation, enzymatic processes, chemical synthesis, or combinations thereof.

Suitable sialylated HMOs for inclusion in the infant formula include at least one sialic acid residue in the oligosaccharide backbone. In certain aspects, the sialylated HMO includes two or more sialic acid residues. Specific non-limiting examples of sialylated HMOs for use in the present disclosure include sialyl oligosaccharides, sialic acid (e.g., free sialic acid, lipid-bound sialic acid, protein-bound sialic acid), lactosialotetraose, 3′-Sialyl-3-fucosyllactose, Disialomonofucosyllacto-N-neohexaose, Monofucosylmonosialyllacto-N-octaose (sialyl Lea), Sialyllacto-N-fucohexaose II, Disialyllacto-N-fucopentaose II, Monofucosyldisialyllacto-N-tetraose), sialyl fucosyl oligosaccharides, 2′-Sialyllactose, 2-Sialyllactosamine, 3′-Sialyllactose, 3′-Sialyllactosamine, 6′-Sialyllactose, 6′-Sialyllactosamine, Sialyllacto-N-neotetraose c, Monosialyllacto-N-hexaose, Disialyllacto-N-hexaose I, Monosialyllacto-N-neohexaose I, Monosialyllacto-N-neohexaose II, Disialyllacto-N-neohexaose, Disialyllacto-N-tetraose, Disialyllacto-N-hexaose II, Sialyllacto-N-tetraose a, Disialyllacto-N-hexaose I, Sialyllacto-N-tetraose b, sialyl-lacto-N-tetraose a, sialyl-lacto-N-tetraose b, sialyl-lacto-N-tetraose c, sialyl-fucosyl-lacto-N-tetraose I, sialyl-fucosyl-lacto-N-tetraose II, disialyl-lacto-N-tetraose, and combinations thereof.

Specific non-limiting examples of fucosylated HMOs for inclusion in the infant formula include fucosyl oligosaccharides, Lacto-N-fucopentaose I, Lacto-N-fucopentaose II, 3′-Fucosyllactose, Lacto-N-fucopentaose III, Lacto-N-difucohexaose I, Lactodifucotetraose, monofucosyllacto-N-hexaose II, isomeric fucosylated lacto-N-hexaose (1), isomeric fucosylated lacto-N-hexaose (3), isomeric fucosylated lacto-N-hexaose (2), difucosyl-para-lacto-N-neohexaose, difucosyl-para-lacto-N-hexaose, difucosyllacto-N-hexaosemonofucosyllacto-neoocataose, monofucosyllacto-N-ocataose, difucosyllacto-N-octaose I, difucosyllacto-N-octaose II, difucosyllacto-N-neoocataose II, difucosyllacto-N-neoocataose I, lacto-N-fucopentaose V, lacto-N-decaose, trifucosyllacto-N-neooctaose, trifucosyllacto-N-octaose, trifucosyl-iso-lacto-N-octaose, lacto-N-difuco-hexaose II, and combinations thereof.

In certain aspects, other suitable examples of HMOs for inclusion in the infant formula include lacto-N-hexaose, para-lacto-N-hexaose, lacto-N-neohexaose, para-lacto-N-neohexaose, lacto-N-neoocataose, para-lacto-N-octanose, iso-lacto-N-octaose, lacto-N-octaose, and combinations thereof.

In certain aspects, the sialylated and fucosylated HMOs (exclusive of the at least one of 2′-FL and the LNnT) are present in the infant formula in a total amount of human milk oligosaccharide in the composition (mg of human milk oligosaccharide per mL of composition as consumed/administered or as suitable for consumption/administration) of at least about 0.001 mg/mL, at least about 0.01 mg/mL, from about 0.001 mg/mL to about 20 mg/mL, from about 0.01 mg/mL to about 20 mg/mL, from 0.001 mg/mL to about 10 mg/mL, from about 0.01 mg/mL to about 10 mg/mL, from 0.001 mg/mL to about 5 mg/mL, from about 0.01 mg/mL to about 5 mg/mL, from about 0.001 mg/mL to about 1 mg/mL, from about 0.001 mg/mL to about 0.23 mg/mL, or from about 0.01 mg/mL to about 0.23 mg/mL of total human milk oligosaccharide in the formula. In certain aspects, the amount of specific sialylated human milk oligosaccharide, the fucosylated human milk oligosaccharide, or both (exclusive of the at least one of the 2′-FL and the LNnT) present in the composition will depend on the specific human milk oligosaccharide or human milk oligosaccharides present and the amounts of other components in the infant formulas, including the amounts of any optional human milk oligosaccharides. Synthetic (i.e., non-human or animal sourced) HMOs are preferably used in the infant formula disclosed herein.

### Polyunsaturated Fatty Acids (PUFAs)

The infant formulas described herein include the PUFAs docosahexaenoic acid (DHA) and arachidonic acid (AA). Research has shown multiple roles for PUFAs in supporting brain and vision development in infants. It is applicants' belief that inclusion of DHA and AA in the infant formula can improve neurological functions, such as cognition, learning, and memory, associated with the CNS.

DHA is an n-3 PUFA and is abundant in the brain and retina, accounting for 40% of the PUFAs in the brain and 60% of the PUFAs in the retina. AA is an n-6 PUFA that is present in the phospholipids, especially phosphatidylethanolamine, phosphatidylcholine, and phosphatidylinositides, of membranes of the body's cells, and is abundant in the brain, muscles, and liver. Given the diverse roles of these PUFAs, it is desirable that intake be balanced in both n-6 and n-3 PUFAs. Thus, the infant formulas disclosed herein include combinations of DHA and AA such that the AA and DHA n-6 to n-3 ratio is 1 to 1.8, including ratios of 1.2 to 1.7, and including a ratio of 1.5.

In certain aspects, the PUFAs are provided as free fatty acids, in triglyceride form, in diglyceride form, in monoglyceride form, in phospholipid form, or as a mixture of one or more of the above, preferably in triglyceride form.

In certain aspects, the infant formula includes at least about 20 mg/L of one or both of DHA and AA. In certain aspects, the infant formulas include DHA in a concentration of at least 60 mg/L, including at least 70 mg/L, including at least 80 mg/L, including at least 90 mg/L, including at least 100 mg/L, including at least 150 mg/L, including at least 200 mg/L and including from 60 mg/L to about 1000 mg/L, and including from about 100 mg/L to about 500 mg/L. In certain aspects, the infant formulas include AA in a concentration of at least 110 mg/L, including at least 120 mg/L, including at least 130 mg/L, including at least 140 mg/L, including at least 150 mg/L, including at least 200 mg/L, and including from 110 mg/L to about 1000 mg/L, and also including from about 110 mg/L to about 500 mg/L.

In addition to the DHA and AA, in certain aspects, the infant formula includes one or more additional PUFAs. Such additional PUFAs include, but are not limited to, eicosapentaenoic acid (EPA), docosapentaenoic acid (DPA), linoleic acid, linolenic acid (alpha linolenic acid) and gamma-linolenic acid. The PUFAs may be derived from oil sources such as plant oils, marine plankton, fungal oils, and fish oils. In certain aspects, the PUFAs are derived from fish oils such as menhaden, salmon, anchovy, cod, halibut, tuna, or herring oil.

To reduce potential side effects of high dosages of PUFAs in the infant formulas, in some aspects, the content of PUFAs does not exceed 3% by weight of the total fat content, including below 2% by weight of the total fat content, and including below 1% by weight of the total fat content in the infant formula.

### Nucleotides

The infant formulas described herein include nucleotides, including, at least, the nucleotide inosine monophosphate. Research has shown nucleotides play a role in protecting individuals from infection. It is applicants' belief that inclusion of nucleotides, including, at least, inosine monophosphate in the infant formula can improve neurological functions, such as cognition, learning, and memory, associated with the CNS. The infant formulas disclosed herein include at least 30 mg/L of nucleotides, including at least 50 mg/L, and at least 100 mg/L of nucleotides.

The nucleotide concentration in the infant formula is preferably from the inosine monophosphate alone, or, in certain aspects, is from the inosine monophosphate in combination with one or more additional nucleotides, nucleotide precursors, or both. Non-limiting examples of nucleotide precursors include nucleosides, purine bases, pyrimidine bases, ribose and deoxyribose. The nucleotide may be in monophosphate, diphosphate, or triphosphate form. The nucleotide may be a ribonucleotide or a deoxyribonucleotide. The nucleotides may be monomeric, dimeric, or polymeric (including RNA and DNA). The nucleotide may be present in the nutritional composition as a free acid or in the form of a salt, preferably a monosodium salt.

Non-limiting examples of additional nucleotides and nucleosides for use in the infant formulas include one or more of cytidine 5′-monophosphate, uridine 5′-monophosphate, adenosine 5′-monophosphate, guanosine 5′-1-monophosphate, more preferably cytidine 5′-monophosphate, uridine 5′-monophosphate, adenosine 5′-monophosphate, and guanosine 5′-monophosphate.

### Carotenoids

The infant formulas described herein additionally include lutein. Lutein has been shown to provide oxidative protection, as well as to enhance brain development of the infant. It is applicants' belief that inclusion of lutein in the infant formula can improve neurological functions, such as cognition, learning, and memory, associated with the CNS. The lutein is present in the infant formula at a concentration of at least 30 μg/L, including at a concentration between 44 μg/L and 5 mg/L.

In certain aspects, the infant formulas contain trans-lutein. As used herein, “trans-lutein” refers to a compound having the following structure:

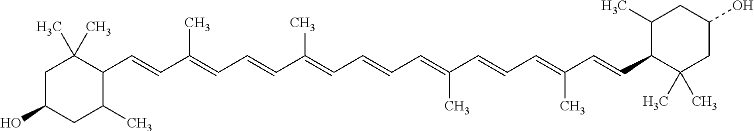 [SMILES: CC(/C=C/C=C(C)/C=C/C1C(C)C[C@@H](O)CC1(C)C)=C\C=C\C=C(C)\C=C\C=C(C)\C=C\[C@H]1C(C)C[C@H](O)CC1(C)C]

In those aspects where the infant formula includes trans-lutein, the infant formula can contain trans-lutein in combination with other lutein forms, or, in some aspects, the lutein is in all-trans form. In certain aspects, the lutein is at least 50% trans-lutein, including at least 60% trans-lutein, including at least 70% trans-lutein, including at least 80% trans-lutein, including at least 90% trans-lutein, and in certain aspects, including at least 95% trans-lutein.

In certain aspects, the infant formulas include one or more additional carotenoids such as beta-carotene, zeaxanthin, lycopene, and combinations thereof. It should be understood that any combination of beta-carotene, zeaxanthin, and lycopene can be included along with lutein in the infant formulas of the present disclosure. In those aspects where lycopene is included in the infant formula, lycopene is preferably present in a concentration of from 0.001 μg/mL to 5 μg/mL, from 0.001 μg/mL to 0.0130 μg/mL, including from 0.001 μg/mL to 0.0075 μg/mL of lycopene, and also including from 0.0185 μg/L to 5 μg/L of lycopene. In those aspects where beta-carotene is included in the infant formula, beta-carotene is preferably present in a concentration of from 1 μg/mL to 5 μg/mL, including from 0.001 μg/mL to 0.025 μg/L of beta-carotene, including from 0.001 μg/L to 0.011 μg/mL of beta-carotene, and also including from 0.034 μg/mL to 5 μg/mL of beta-carotene.

Other carotenoids may optionally be included in the infant formulas as described herein. Any or all of the carotenoids included in the infant formulas described herein may be from a natural source, or artificially synthesized.

Each of the carotenoids in the selected combinations can be obtained from any known or otherwise suitable material source for use in infant formulas, and each can be provided individually, or all together, or in any combination and from any number of sources, including sources such as multivitamin premixes containing other vitamins or minerals in combination with one or more of the carotenoids as described herein. Non-limiting examples of some suitable sources of lutein, lycopene, beta-carotene, or combinations thereof include LycoVit® lycopene (available from BASF, Mount Olive, N.J.), Lyc-O-Mato® tomato extract in oil, powder, or bead form (available from LycoRed Corp., Orange, N.J.), beta-carotene, lutein, or lycopene (available from DSM Nutritional Products, Parsippany, N.J.), FloraGLO® lutein (available from Kemin Health, Des Moines, Iowa), Xangold® Natural Lutein Esters (available from Cognis, Cincinnati, Ohio), and Lucarotin® beta-carotene (available from BASF, Mount Olive, N.J).

### RRR-Alpha-Tocopherol

In certain aspects, the infant formulas described herein include RRR-alpha-tocopherol. Brain analyses show a correlation between cholesterol, glutamate, and RRR-alpha-tocopherol. Cholesterol is a major component of myelin and is a marker for extent of CNS myelination. Glutamate is a neurotransmitter, which is reported to stimulate neuron outgrowth and branching. Research suggests that RRR-alpha-tocopherol stimulates newborn infant CNS development. It is applicants' belief that inclusion of RRR-alpha-tocopherol in the infant formula can improve neurological functions, such as cognition, learning, and memory, associated with the CNS.

In certain aspects, the RRR-alpha-tocopherol is present in the infant formula in an amount sufficient to improve brain or CNS development. In some aspects, the infant formulas contain RRR-alpha-tocopherol in concentrations of at least about 5 mg/L, including at least about 7 mg/L, including at least about 8 mg/L, including at least about 9 mg/L, including at least about 10 mg/L, including at least about 15 mg/L, including at least about 18 mg/L, including at least about 20 mg/L, including from at least about 5 mg/L to about 100 mg/L, including from at least about 7 mg/L to about 50 mg/L, and including from about 20 mg/L to about 40 mg/L. The total amounts of RRR-alpha-tocopherol include both exogenous and inherent sources of RRR-alpha-tocopherol, as noted below.

As used herein, the term “RRR-alpha-tocopherol” refers both to exogenous sources and inherent sources of RRR-alpha-tocopherol and RRR-alpha-tocopherol acetate that are present in an infant formula. Inherent sources include RRR-alpha-tocopherol that is inherently present in components that are present in a infant formula and may include for example, various oils and fats. Exogenous sources of RRR-alpha-tocopherol include RRR-alpha-tocopherol that is added to the infant formula not as part of another component.

Tocopherols, generically referred to as vitamin E, are available in four forms: alpha-, beta-, gamma-, and delta-, which differ in the number and position of the methyl groups on the chroman ring, as shown by the structure below and Table 1.

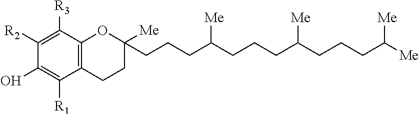

TABLE 1
Structure of Natural Tocopherols

| Compound | R₁ | R₂ | R₃ |
|---|---|---|---|
| alpha-tocopherol | Me | Me | Me |
| beta-tocopherol | Me | H | Me |
| gamma-tocopherol | H | Me | Me |
| delta-tocopherol | H | H | Me |

Tocopherols can exist in a number of stereoisomeric forms depending on the chirality of the phytyl tail. Of the alpha-tocopherols, RRR-alpha-tocopherol (also referred to as “natural vitamin E”) has the greatest biological activity and is reported to be the dominant form of alpha-tocopherol in the brain. RRR-alpha-tocopherol is a single stereoisomer whereas synthetic vitamin E (all-rac-alpha-tocopherol or tocopherol acetate) is an equimolar mixture of eight isomers, only one of which is RRR-alpha-tocopherol. The fact that the dominant form of alpha-tocopherol is RRR-alpha-tocopherol (based on animal studies) strongly suggests that the other seven chiral isomers are absorbed at a lower rate by the brain or oxidized at a faster rate.

It has been reported that the RRR-alpha-tocopherol content and the alpha- to gamma-tocopherol ratio of human milk decrease with the stage of lactation. The alpha- to gamma-tocopherol ratio of colostrum is about 10 and it decreases to about 4-6 in mature milk. It is possible that these decreasing alpha- to gamma-tocopherol ratios are optimized for maximum protection of infants from oxidative stress without adversely affecting infant CNS development. Gamma-tocopherol has been shown to be negatively correlated with phospholipid content. Gamma-tocopherol, has, however, been shown to serve as an antioxidant.

In certain aspects, it is desirable that the infant formula also contain gamma-tocopherol, but that the amount of gamma-tocopherol, the ratio of RRR-alpha-tocopherol to gamma-tocopherol, or both, be controlled. Accordingly, when present, the gamma-tocopherol is desirably present in the infant formulas in concentrations of less than 7 mg/L, including less than 5 mg/L, including from 0 mg/L to 3 mg/L, including from about 1 mg/L to 3 mg/L of the infant formula. Alternatively or additionally, potential negative effects of gamma-tocopherol are controlled by including RRR-alpha-tocopherol in excess. For example, in certain aspects, the alpha- to gamma-tocopherol ratio is from about 2:1 to about 20:1, or from about 3.5:1 to about 15:1; or from about 5:1 to about 10:1. Vitamin E or a source thereof having an alpha- to gamma-tocopherol ratio from about 2:1 to about 20:1 is also referred to herein as “an optimized tocopherol blend.”

In addition to balancing n-6 to n-3 PUFAs described above, where the infant formulas include RRR-alpha-tocopherol, it is desirable that the amount of RRR-alpha-tocopherol, DHA and AA be present in particular weight ratios. As such, in certain aspects, the weight ratio of DHA to RRR-alpha tocopherol ranges from about 5:1 to about 15:1, desirably from about 7.5:1 to about 10:1, and the weight ratio of AA to RRR-alpha tocopherol ranges from about 12:1 to about 24:1, desirably from about 12:1 to about 18:1.

### Macronutrients

In certain aspects, the infant formula will include at least one of protein, fat, and carbohydrate. In many aspects, the infant formula will include protein, fat, and carbohydrate.

Where present, carbohydrate concentrations most typically will range from about 5% to about 40%, including from about 7% to about 30%, including from about 10% to about 25%, by weight of the infant formula. Where present, fat concentrations most typically range from about 1% to about 30%, including from about 2% to about 15%, and also including from about 3% to about 10%, by weight of the infant formula. Where present, protein concentrations most typically range from about 0.5% to about 30%, including from about 1% to about 15%, and also including from about 2% to about 10%, by weight of the infant formula.

The amount of any or all of the carbohydrates, fats, and proteins in any of the infant formula described herein may also be characterized as a percentage of total calories in the infant formula as set forth in the following table. These macronutrients for infant formulas of the present disclosure are most typically formulated within any of the caloric ranges (embodiments A-F) described in the following table (each numerical value is preceded by the term “about”).

TABLE 2
Exemplary macronutrient profiles of the infant formula

| | Embodiment A | Embodiment B | Embodiment C |
|---|---|---|---|
| Nutrient | (% Total Cal.) | (% Total Cal.) | (% Total Cal.) |
| Carbohydrate | 0-98 | 2-96 | 10-75 |
| Protein | 0-98 | 2-96 | 5-70 |
| Fat | 0-98 | 2-96 | 20-85 |
| | Embodiment D | Embodiment E | Embodiment F |
| | (% Total Cal.) | (% Total Cal.) | (% Total Cal.) |
| Carbohydrate | 30-50 | 25-50 | 25-50 |
| Protein | 15-35 | 10-30 | 5-30 |
| Fat | 35-55 | 1-20 | 2-20 |

### Fat

In some aspects, the infant formulas of the present disclosure include a source or sources of fat in addition to the PUFAs, described above. Suitable sources of fat for use herein include any fat or fat source that is suitable for use in an oral infant formula and is compatible with the essential elements and features of such formula.

Additional non-limiting examples of suitable fats or sources thereof for use in the infant formulas described herein include coconut oil, fractionated coconut oil, soybean oil, corn oil, olive oil, safflower oil, high oleic safflower oil, oleic acids (EMERSOL 6313 OLEIC ACID, Cognis Oleochemicals, Malaysia), MCT oil (medium chain triglycerides), sunflower oil, high oleic sunflower oil, palm and palm kernel oils, palm olein, canola oil, marine oils, fish oils, fungal oils, algae oils, cottonseed oils, and combinations thereof.

### Protein

In certain aspects, the infant formulas of the present disclosure include protein. Any protein source that is suitable for use in oral infant formulas and is compatible with the essential elements and features of such formulas is suitable for use in the infant formulas.

Non-limiting examples of suitable proteins or sources thereof for use in the infant formulas include hydrolyzed, partially hydrolyzed or non-hydrolyzed proteins or protein sources, which may be derived from any known or otherwise suitable source such as milk (e.g., casein, whey), animal (e.g., meat, fish), cereal (e.g., rice, corn), vegetable (e.g., soy) or combinations thereof. Non-limiting examples of such proteins include milk protein isolates, milk protein concentrates as described herein, casein protein isolates, extensively hydrolyzed casein, whey protein, sodium or calcium caseinates, whole cow milk, partially or completely defatted milk, soy protein isolates, and soy protein concentrates. In an aspect, the infant formulas include a protein source derived from milk proteins of human and/or bovine origin.

In an aspect, the protein source is a hydrolyzed protein, i.e., a protein hydrolysate. In this context, the terms “hydrolyzed protein” or “protein hydrolysates” are used interchangeably herein and include extensively hydrolyzed proteins, wherein the degree of hydrolysis is most often at least about 20%, including from about 20% to about 80%, and also including from about 30% to about 80%, even more preferably from about 40% to about 60%. The degree of hydrolysis is the extent to which peptide bonds are broken by a hydrolysis method. The degree of protein hydrolysis for purposes of characterizing the extensively hydrolyzed protein component of these embodiments is easily determined by one of ordinary skill in the formulation arts by quantifying the amino nitrogen to total nitrogen ratio (AN/TN) of the protein component of the selected liquid formulation. The amino nitrogen component is quantified by USP titration methods for determining amino nitrogen content, while the total nitrogen component is determined by the Tecator Kjeldahl method, all of which are well known methods to one of ordinary skill in the analytical chemistry art.

Suitable hydrolyzed proteins include soy protein hydrolysate, casein protein hydrolysate, whey protein hydrolysate, rice protein hydrolysate, potato protein hydrolysate, fish protein hydrolysate, egg albumen hydrolysate, gelatin protein hydrolysate, combinations of animal and vegetable protein hydrolysates, and combinations thereof. Particularly preferred protein hydrolysates include whey protein hydrolysate and hydrolyzed sodium caseinate.

When used in the infant formulas, the protein source may include at least about 20% (by weight total protein) protein hydrolysate, including from about 30% to 100% (by weight total protein) protein hydrolysate, and including from about 40% to about 80% (by weight total protein) protein hydrolysate, and including about 50% (by weight total protein) protein hydrolysate. In an aspect, the infant formula includes 100% (by weight total protein) protein hydrolysate.

### Carbohydrate

In some aspects, the infant formulas of the present disclosure include carbohydrates that are suitable for use in an oral infant formula and are compatible with the essential elements and features of such formulas.

Non-limiting examples of suitable carbohydrates or sources thereof for use in the infant formulas described herein include maltodextrin, hydrolyzed or modified starch or cornstarch, glucose polymers, corn syrup, corn syrup solids, rice-derived carbohydrates, pea-derived carbohydrates, potato-derived carbohydrates, tapioca, sucrose, glucose, fructose, lactose, high fructose corn syrup, honey, sugar alcohols (e.g., maltitol, erythritol, sorbitol), artificial sweeteners (e.g., sucralose, acesulfame potassium, stevia) and combinations thereof. A particularly desirable carbohydrate is a low dextrose equivalent (DE) maltodextrin.

### Methods of Manufacture

The infant formulas of the present disclosure may be prepared by any known or otherwise effective manufacturing technique for preparing the formula. Many such techniques are known for any given product form such as nutritional liquids or powders and can easily be applied by one of ordinary skill in the art to the infant formulas described herein.

The infant formulas of the present disclosure can therefore be prepared by any of a variety of known or otherwise effective formulation or manufacturing methods. In one suitable manufacturing process, for example, at least three separate slurries are prepared, including a protein-in-fat (PIF) slurry, a carbohydrate-mineral (CHO-MIN) slurry, and a protein-in-water (PIW) slurry. The PIF slurry is formed by heating and mixing the oil (e.g., canola oil, corn oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra trace minerals (TM/UTM premix), thickening or suspending agents (e.g., avicel, gellan, and carrageenan). The resulting CHO-MIN slurry is held for 10 minutes with continued heat and agitation before adding additional minerals (e.g., potassium chloride, magnesium carbonate, potassium iodide, etc.), and/or carbohydrates (e.g., HMOs, fructooligosaccharide, sucrose, corn syrup, etc.). The PIW slurry is then formed by mixing with heat and agitation the remaining protein, if any.

The resulting slurries are then blended together with heated agitation and the pH adjusted to 6.6-7.0, after which the composition is subjected to high-temperature short-time (HTST) processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level. The formula is then aseptically packaged to form an aseptically packaged nutritional emulsion. This emulsion can then be further diluted, heat-treated, and packaged to form a ready-to-feed or concentrated liquid, or it can be heat-treated and subsequently processed and packaged as a reconstitutable powder, e.g., spray dried, drymixed, agglomerated.

The nutritional solid, such as a spray dried nutritional powder or drymixed nutritional powder, may be prepared by any collection of known or otherwise effective techniques, suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising predigested fat, and optionally protein, carbohydrate, and other sources of fat, and then spray drying the slurry or liquid to produce a spray dried nutritional powder. The method may further comprise the step of spray drying, drymixing, or otherwise adding additional nutritional ingredients, including any one or more of the ingredients described herein, to the spray dried nutritional powder.

Other suitable methods for making infant formulas are described, for example, in U.S. Pat. No. 6,365,218 (Borschel, et al.), U.S. Pat. No. 6,589,576 (Borschel, et al.), U.S. Pat. No. 6,306,908 (Carlson, et al.), U.S. Patent Application No. 20030118703 A1 (Nguyen, et al.), which descriptions are incorporated herein by reference to the extent that they are consistent herewith.

### Methods of Use

The methods of use of the present disclosure include the oral administration of the infant formulas that include PUFAs, nucleotides, lutein, and at least one of 2′-FL and LNnT to improve at least one of cognition, learning, and memory in an infant. Particularly, the disclosed combination of PUFAs, nucleotides, lutein, and at least one of 2′-FL and LNnT, may improve general cognition by enhancing memory acquisition, memory retention and memory recall that contributes to the cognitive functions of learning, thinking, and memory

The infant formulas as described herein can be administered to individuals including infants generally, or may, in some aspects, be administered to a specific subclass of infants that are “in need thereof;” that is, to specific infants that would specifically benefit by administration of the infant formula. For example, a specific infant may be “in need of” the infant formulas as described herein if they are susceptible to (i.e., have one or more of a genetic predisposition, a family history of, and symptoms of the disease or condition) neurodegenerative diseases or other diseases and conditions that can impair/reduce cognition generally or specific aspects of cognition.

The individual desirably consumes at least one serving of the infant formula daily, and in some embodiments, may consume two, three, or even more servings per day. Each serving is desirably administered as a single, undivided dose, although the serving may also be divided into two or more partial or divided servings to be taken at two or more times during the day. The methods of the present disclosure include continuous day after day administration, as well as periodic or limited administration, although continuous day after day administration is generally desirable. The methods of the present disclosure are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least about 18-24 months, desirably as a long term, continuous, daily, dietary source or supplement.

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described. Accordingly, departures may be made from such details without departing from the spirit or scope of the applicant's general disclosure herein.

1. An infant formula comprising:
at least one human milk oligosaccharide selected from 2′-fucosyllactose (2′-FL) and lacto-N-neotetraose (LNnT);
polyunsaturated fatty acids (PUFAs) docosahexaenoic acid (DHA) and arachidonic acid (AA), wherein the AA and DHA are present in an n-6/n-3 ratio of between 1 and 1.8;
at least 30 mg/L of nucleotides, wherein the nucleotides comprise inosine monophosphate; and
at least 30 μg/L of lutein.

2. The infant formula of claim 1 comprising between about 0.001 mg/mL and about 20 mg/mL of the at least one human milk oligosaccharide.

3. The infant formula of claim 1 wherein the infant formula comprises from about 0.5 mg/mL to about 1 mg/mL of the at least one human milk oligosaccharide.

4. The infant formula of claim 1 wherein the PUFAs are present in the infant formula as free fatty acids, in triglyceride form, in diglyceride form, in monoglyceride form, in phospholipid form, or as a mixture thereof.

5. The infant formula of claim 1 wherein the infant formula comprises at least about 20 mg/L of PUFAs.

6. The infant formula of claim 1 wherein the lutein is at least 50% trans-lutein.

7. The infant formula of claim 1 wherein the infant formula further comprises at least one of a carbohydrate, a protein, and a fat.

8. The infant formula of claim 1 wherein the PUFAs DHA and AA are derived from fish oil.

9. The infant formula of claim 1 further comprising RRR-alpha-tocopherol.

10. A method for improving at least one of cognition, learning, and memory in an infant, the method comprising administering to the infant an infant formula comprising at least one human milk oligosaccharide selected from 2′-FL and LNnT;
PUFAs DHA and AA, wherein the AA and DHA are present in an n-6/n-3 ratio of between 1 and 1.8;
at least 30 mg/L of nucleotides, wherein the nucleotides comprise inosine monophosphate; and
at least 30 μg/L of lutein.

11. The method of claim 10 wherein the infant formula comprises between about 0.001 mg/mL and about 20 mg/mL of the at least one human milk oligosaccharide.

12. The method of claim 10 wherein the infant formula comprises from about 0.5 mg/mL to about 1 mg/mL of the at least one human milk oligosaccharide.

13. The method of claim 10 wherein the PUFAs are present in the infant formula as free fatty acids, in triglyceride form, in diglyceride form, in monoglyceride form, in phospholipid form, or as a mixture thereof.

14. The method of claim 10 wherein the infant formula comprises at least about 20 mg/L of PUFAs.

15. The method of claim 10 wherein the lutein is at least 50% trans-lutein.

16. The method of claim 10 wherein the infant formula further comprises at least one of a carbohydrate, a protein, and a fat.

17. The method of claim 10 wherein the PUFAs DHA and AA are derived from fish oil.

18. The method of claim 10 wherein the infant formula further comprises RRR-alpha-tocopherol.

19. A composition for use in for improving at least one of cognition, learning, and memory in an individual, the composition comprising the combination of:
at least one human milk oligosaccharide selected from 2′-fucosyllactose (2′-FL) and lacto-N-neotetraose (LNnT);
polyunsaturated fatty acids (PUFAs) docosahexaenoic acid (DHA) and arachidonic acid (AA), wherein the AA and DHA are present in an n-6/n-3 ratio of between 1 and 1.8;
at least 30 mg/L of nucleotides, wherein the nucleotides comprise inosine monophosphate; and
at least 30 μg/L of lutein.
